(19) United States
(12) Patent Application Publication
(10) Pub. No.: US 2004/0088745 A1
(43) Pub. Date: May 6, 2004

(54) Production of mammals which produce progeny of a single sex

(76) Inventors: James Robl, Belchertown, MA (US); F. Abel Ponce De Leon, North Oaks, MN (US)

Correspondence Address: MERCHANT & GOULD PC, P.O. BOX 2903, MINNEAPOLIS, MN, 55402-0903, US

(21) Appl. No.: 10/376,735

(22) Filed: Feb. 25, 2003

Related U.S. Application Data
(63) Continuation of application No. PCT/US01/05932, filed on Feb. 26, 2001.
(60) Provisional application No. 60/184,830, filed on Feb. 24, 2000.

(51) Int. Cl. A01K 67/027; C12N 15/85

(52) U.S. Cl. 800/15; 435/455

(57) ABSTRACT

Disclosed are methods of genetically modifying animals such that the animals will produce offspring or progeny of a single sex.

### BACKGROUND OF THE INVENTION

1. Field of the Invention

This invention is directed generally to methods for producing offspring of a single sex. In contrast to the tedious sperm-separation methods used by others in the past, the methods of the invention are accomplished using genetic modification of the germ line. Accordingly, the trait of producing a single type of progeny may be passed on to subsequent generations. The technology has particular applicability in the field of agriculture, and particularly in the beef and swine industries.

2. Technology Background

All publications and patent applications herein are incorporated by reference to the same extent as if each individual publication or patent application was specifically and individually indicated to be incorporated by reference.

Mammalian males and females are distinguishable genetically by the identity of the sex chromosomes. Normal female mammals contain two X chromosomes, whereas normal males contain one X and one Y. Since female mammals can donate only an X chromosome during mating, it is the male gamete which determines the sex of the offspring. Because mammalian semen normally contains approximately equal numbers of X-chromosome and Y-chromosome bearing sperm, the chances of having either a male or female offspring as a result of normal mating techniques is very close to fifty percent.

The ability to alter the probability of having an offspring of a particular sex has been the subject of much interest over the past couple decades. In human reproduction, such interests have been fueled in part by the desire to reduce the incidence of sex-linked disorders. For instance, there are hundreds of X-linked diseases which are typically manifested in males since males only receive one X-chromosome, e.g., hemophilia, Lesch-Nyhand syndrome. By increasing the chances of having a female, a couple can avoid having a male child who exhibits such a disorder.

In the agricultural industry, the ability to pre-determine the sex of livestock has the potential to improve both the economics and management of the industry. Most livestock farmers place a premium on animals based on their sex, depending on the particular sector of the industry. For instance, dairy farmers have essentially no use for male calves. Beef farmers, on the other hand, prefer male calves, which grow faster and gain weight more efficiently than their female counterparts. Swine farmers prefer the bacon produced using female pigs over that of males. And poultry farms would likely choose hens more often than roosters.

Methods to mechanically sort sperm by a variety of methods have been available for some time, which have led to the commercial sale of semen preparations which may be used in artificial insemination to affect the sex of offspring. U.S. Pat. Nos. 5,439,362 and 5,840,504 provide a review of the various mechanical approaches, and are herein incorporated by reference. Such approaches have included techniques based on the characteristics of the sperm, e.g., size, head shape, mass, surface properties, surface macromolecules, DNA content, swimming velocity, and motility (see review by Windsor et al., 1993, Reprod. Fert. Dev. 5:155). For instance, attempts to separate sperm by immunological methods based on potential differences in membrane antigen profiles have also been made (e.g., U.S. Pat. No. 5,439,362). More recent methods have focused on sperm sorting techniques, whereby sperm cells are treated with a fluorescent dye and sorted using flow cytometry based on the higher DNA content, and accordingly the higher fluorescence of the X-carrying sperm (Johnson, 1996, Gender preselection in mammals: an overview, DTW 103(8-9): 288-291).

However, mechanical separation processes are tedious and not entirely accurate. With sperm sorting techniques in particular, the inability to obtain large numbers of sperm in a short amount of time would complicate the use of such sperm in artificial insemination procedures. Moreover, some have argued that the labeling of the DNA has the potential to cause genetic damage. Also, mechanical sperm sorting techniques offer no possibility of carrying specific traits through an individually bred line of single sex animals, and a farmer wishing to manipulate the sex of an animal's offspring must purchase sperm for every insemination.

Embryo separation techniques have been most successful, whereby embryos are recovered from the mother, a biopsy of the cells is taken, and PCR amplification is used to analyze sex chromosome-specific DNA. However, this approach is very tedious, requires extensive training, requires expensive equipment, and requires a recipient female into which the embryo may be transferred. As a result, the technique is rarely used.

There have been few reports on genetic modifications of the germ line that aim to bias reproduction to favor offspring of a particular sex. U.S. Pat. No. 5,596,089 reports a method of manipulating the sex phenotype of mammals by using the SRY promoter (from the y-chromosome encoded testes determining factor) to initiate transcription of a diptheria toxin gene in male gonadal tissue during embryonic development. The gene is controlled and activated using the Cre-Lox system. However, this method manipulates the sexual phenotype only, as the female offspring resulting from such a manipulation would still be genetically male (XY).

U.S. Pat. No. 5,223,610 of Burton et al. suggests that creating transgenic animals which express a non-lethal modulator in spermatids might one be a way to test the effect of therapeutics on sperm fertility. Although alterations in spermatogenesis are a predicted outcome, Burton et al. do not suggest that such a process might be used to manipulate the sex of the resulting offspring.

The present invention provides several advantages while also overcoming the deficiencies of the prior art. Firstly, after the transgenic animals of the present invention are created, there is no need for further technology to produce offspring of a particular sex. A male giving rise to single sex offspring could be used in multiple normal matings or in artificial insemination protocols. Furthermore, when a male transgenic mammal is used to create the single sex offspring, the genetic modification is not passed on to subsequent generations and the proprietary nature of the invention is protected. Once the genetic modification is developed, it may be propagated at a relatively low cost by cloning techniques, or even natural breeding techniques using a carrier female.

### 3. SUMMARY OF THE INVENTION

The present invention relates to methods for producing animals which have an altered tendency to produce progeny of a particular sex, particularly methods for producing mammals having such a tendency. Such methods involve genetically modifying the heterogametic sex (the sex that carries two different sex chromosomes and therefor determines the sex of the offspring), such that the genetically modified gamete is marked or disabled. Such mammals, also a subject of the present invention, will give rise to single sex offspring.

Also included are methods of genetically modifying the homogametic sex such that heterogametic offspring of such animals will give rise to single sex offspring. Such genetically modified homogametic animals, also a subject of the invention, provide a means of propagating the single sex producing trait using breeding techniques. Such breeding techniques are also a subject of the present invention. Also encompassed are the genetic constructs and tools used to accomplish the methods described herein.

### 5. DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. For purposes of the present invention, the following terms are defined below.

As used herein, heterogametic means having two different sex chromosomes, i.e., an X chromosome and a Y chromosome, and therefor indicates that such an animal will determine the sex of the offspring. In mammals, the heterogametic sex is the male, and in birds, it is the female. Homogametic, then, means having two of the same chromosome, i.e., as for genetically normal female mammals which have two X chromosomes.

### DESCRIPTION OF THE INVENTION

The present invention includes a method for producing an animal, particularly a mammal, wherein the animal has an altered tendency to produce progeny of a particular sex. The term “progeny” refers to either direct offspring or descendants, i.e., offspring of offspring, depending on the sex of the animal produced.

Such methods are performed by introducing a nucleic acid construct into at least one sex chromosome of the germ line of said mammal, wherein the nucleic acid construct encodes a transgene which is expressed post-meiotically in developing spermatids. Expression of the transgene is designed to alter the fertility of sperm resulting from said developing spermatids, such that the mammal produced has an altered tendency to produce progeny of a particular sex in a subsequent generation.

The methods may be directed to producing both heterogametic and homogametic animals. For instance, when the methods produce heterogametic sperm-producing animals having the transgene on a sex chromosome, the gamete which carries the transgene after meiosis will have altered fertility, i.e., altered capability to complete fertilization of an egg. Such animals will therefor have an unnatural probability of fostering progeny of a particular sex in the first generation of offspring, the probability depending on the nature of the transgene and the extent to which sperm expressing the transgene are disabled.

When the methods produce homogametic egg-producing animals, the probability of having offspring of a particular sex is not affected in the first generation, because such an animal does not produce sperm. Therefor, if the transgene is on one of the two sex chromosomes, it will be passed to approximately half of the offspring depending on natural probability, whether male or female. If the transgene is on both sex chromosomes, all offspring will receive the transgene. However, the probability of having a particular sex in the first generation progeny from an egg-producing mammal will not be affected if the transgene is designed to affect sperm fertility.

Egg producers which receive the transgene from a transgenic egg-producing parent will carry the line, but since they do not produce sperm, their direct offspring will also not be affected. Sperm-producing heterogametic animals which receive the transgene however, will have substantially single sex offspring to the extent that any sperm acquiring the transgene-bearing chromosome following meiosis is disabled. Since egg-producing homogametic animals have the capability of carrying the line indefinitely, sperm-producers of any subsequent generation may be affected when the transgene is introduced into a line of homogametic animals.

The method of the present invention, whereby animals are produced which have an altered tendency to produce progeny of a particular sex, is basically accomplished by introducing a transgene into the germline of the animal. Accordingly, any technology appropriate for producing transgenic animals may be used. Particularly preferred methods include nuclear transfer technology, described in detail in U.S. Pat. No. 5,945,577, and copending application Ser. Nos. 08/888,057 and 08/888,283, incorporated herein by reference. Of course, once an appropriate transgenic animal is created by introducing the transgene into the germ line of an animal, the transgenic animals of the present invention may be produced using natural breeding techniques.

It is preferable that expression of the transgene merely disable the sperm, for instance, reduce its motility or fertilizing ability, rather than kill the sperm. This would mean that the methods of the present invention could be performed using intracytoplasmic sperm injection into a female donor egg. In this way, homogametic female carrier lines could be regenerated using in vitro techniques and the sperm from transgenic X-chromosome bearing males. Likewise, heterogametic males which produce only female offspring could be produced from the sperm of transgenic Y-bearing males. However, transgenes which exert a toxic affect upon the sperm upon expression may also be used, since the animals of the invention may be readily generated using nuclear transfer or other genetic techniques.

To affect specific expression of the transgene in developing spermatids, expression of the transgene may be controlled by a sperm-specific control sequence. Such a control sequence may affect specific expression in sperm either by transcriptional or translational control mechanisms. In a preferred embodiment, the control sequence is a sperm cell-specific gene promoter, which specifically affects transcription only in post-meiotic spermatids. Many such promoters have been identified, any of which may be used to affect specific expression of the transgene in post-meiotic sperm. In particular, sperm-specific control sequences include the protamine 1 or 2 gene promoters.

The term “altered fertility” or “altered tendency to produce progeny of a particular sex” basically indicates that expression of the transgene affects the developing transgenic spermatid in some manner such that it does not have the same capability to affect fertilization of an egg as does its non-transgenic counterpart. In the preferred embodiments, this is accomplished by disabling the sperm containing transgenic chromosomes such that the non-transgenic sperm have a competitive advantage in the fertilization process. However, embodiments where the transgene itself provides a competitive advantage, i.e., improved sperm motility, are also envisioned.

For transgenes which encode structural proteins, such proteins should have the characteristics of (1) not passing through cytoplasmic junctions between spermatids and, therefore, remaining localized in the spermatid containing the transgenic chromosome and (2) either disabling, marking, or enhancing the fertility of the spermatid containing the transgenic chromosome. With regard to haploid expression of the transgene, it has been argued that spermatids share either gene products or transcripts by way of cytoplasmic bridges during spermatogenesis, making gametes phenotypically diploid during post-meiotic stages of development. However, some studies have shown this is not always the case (e.g., Zheng and Martin-Deleon, 1997, Mol. Repro. Dev. 46: 252-257). In fact, it has been suggested that some gene transcripts become membrane bound or otherwise stably localized immediately after transport from the nucleus, as do transcripts encoding cytoskeletal proteins, and would therefor not be expected to be shared among conjoined spermatids (Caldwell and Handel, 1991, Proc. Natl. Acad. Sci. USA 88: 2407-2411). There are also multiple reports of transcripts being stored in non-polysomal ribonucleoprotein (RNP) particles (Burmester and Hoyer-Fender, 1996, Mol. Repro. Dev. 45: 10-20; Sommerville and Ladomery, 1996, Chromosoma 104: 469-478), or cytoplasmic organelles such as chromatoid bodies (CB) (Biggiogera e al., 1990, Mol. Repro. Dev. 26: 150-158), further suggesting that such transcripts would not readily be passed between spermatids.

But even if transcripts are shared between spermatids, the option to control or bias sperm fertility in favor of one sex could be effectuated via genetic mechanisms. For instance, certain autonomous selfish elements are thought to effect sperm fertility and disequilibrium between the developing spermatids via transcript sharing (e.g., the murine t allele, for a review see Miller, 1997, Mol. Human Repro. 3(8): 669-676). It has been suggested that such selfish elements encode transcripts or gene products which diffuse freely across cytoplasmic bridges, disabling the spermatid carrying the wild type allele, while conferring immunity against the disabling effect on the spermatid which receives the element. By inserting such an element into the Y chromosome, for example, a variety of mating scenarios may be envisioned. For instance, a male engineered to carry the selfish element on the Y chromosome may be mated with a female engineered to carry the wild type allele on both X chromosomes. Such a mating pair will only have male offspring.

If transcript sharing does occur, it may also be possible to anchor the transcripts within the X- or Y-chromosome bearing spermatid using regulatory sequences. For instance, the promoter used in the invention may be a hybrid promoter designed from sequences derived from different sperm-specific control sequences. In particular, it has been shown that binding of a phosphoprotein to the 3 untranslated region of mouse protamine 2 mRNA acts to repress translation of the mRNA until a later stage during spermatogenesis, presumably after the regulatory protein is dephosphorylated (Fajardo et al., 1995, Dev. Biol., 1994, 166: 643-653). A similar means of regulation has been proposed for other sperm-specific genes (Kwon and Hecht, 1993, Mol. Cell. Biol. 13 (10): 6547-6557). Thus, by ensuring the constructs of the present invention contain an appropriate 3′ UTR, it should be possible to anchor transcripts in the haploid spermatid using protein interaction.

For embodiments where the transgene product affects the fertility of the spermatid in which it is located, examples of proteins which may be suitable for the purposes of the invention are the highly insoluble cytoskeletal elements of the sperm making up the outer dense fibers (ODF) or fibrous sheath of the sperm tail or the perinuclear theca in the sperm head. These proteins form large clusters in the spermatid and would likely not pass from one spermatid to another. Another example would be a protein containing a strong nuclear localization sequence that would direct the protein to the nucleus and, therefore, keep the protein from passing from one spermatid to another.

To disable the sperm the transgene product could be over expressed, modified so as not to function correctly, i.e., mutated, or could be from another species. Alteration of cytoskeletal or nuclear proteins could result in sperm with altered and less efficient motility or other defects and lower fertility. To enhance sperm performance, such proteins could conceivably be altered, i.e., beneficial mutations, such that function is enhanced. Nuclear regulatory proteins that play a role in metabolism might also be manipulated to give a competitive advantage when expressed specifically in sperm.

Proteins could also be altered to contain a sequence for a marker protein such as green fluorescent protein that could be used to label spermatids carrying one or the other sex chromosomes, i.e., fusion proteins comprising the protein sequence of green fluorescence protein. The marked sperm could be separated and thus give rise to offspring of only one sex. In addition, fusion proteins to markers such as green fluorescence protein would allow visual monitoring and investigation of protein transfer, if any, through cytoplasmic bridges between spermatids. Such fusion proteins would also allow visual assessment of sperm motility and fertility.

Although the preferred embodiments employ changes in structural proteins to accomplish the methods of the present invention, transgenes which encode transcripts which have a regulatory function, i.e., antisense transcripts, might also be employed to alter sperm fertility when coupled to a sperm-specific control element.

The transgene should generally be inserted into one or the other sex chromosome. For males to be produced in mammals the gene should be inserted into the X-chromosome to disable the X-bearing sperm and for females to be produced the gene should be inserted into the Y-chromosome to disable the Y-bearing sperm. Alternatively, transgenes designed to confer a competitive advantage should be inserted into the sex chromosome that is determinative for the particular progeny sex desired.

To ensure optimal expression of the transgene, it may be useful to insert the gene near an endogenously expressed gene. Genes specifically located on the X and Y chromosomes have been identified and are known in the art. (See U.S. Pat. Nos. 5,595,089, 5,700,926 and 5,763,166, herein incorporated by reference.) Alternatively, a new locus for insertion may be identified using the techniques described below, or other techniques commonly used in the art.

It should be noted that, for transgenes conferring a competitive advantage, embodiments are envisioned where the transgene is inserted next to a gene encoding a desirable trait, which is located on a chromosome other than a sex chromosome (an autosome). The transgene is inserted such that the transgene and the desirable trait are inherited in a linked manner. Accordingly, a spermatid receiving the advantage-conferring transgene on an autosome would also receive the desirable trait in a linked manner, and confer a selective advantage for the propagation of the desirable trait in progeny animals by virtue of the linked, sperm-specific competitive advantage. Such techniques would be helpful for breeders in designing or propagating a line of animals with various desirable traits, foregoing the time and inconvenience of breeding each trait to homozygosity.

The present invention also encompasses transgenic animals produced by the above described methods. With regard to disabling transgenes, transgenic mammals may constitute a line of carrier females which may be propagated by a licensed breeder.

Methods of using transgenic animals according to the invention in methods for substantially altering the natural probability of producing progeny of a particular sex are also encompassed herein. Such a method may be accomplished by breeding a transgenic animal according to the invention using natural breeding techniques such that the natural probability of producing progeny of a particular sex is substantially altered in any successive generation.

Nucleic acid constructs which may be used to accomplish the disclosed methods are also part of the invention. As described above, such a nucleic acid construct comprises a sperm-specific control sequence operably linked to a transgene sequence encoding a protein selected from the group consisting of sperm structural proteins, mutated versions thereof, and fusion proteins designed therefrom. The transgene sequence may be a cDNA sequence, genomic sequence, or artificial sequence.

Vectors comprising such nucleic acid constructs are also included, as are prokaryotic and eukaryotic cell lines comprising either the nucleic acid construct inserted into the chromosome, or a vector carrying the nucleic acid construct. Particularly useful cell lines include a fibroblast cell line comprising the nucleic acid construct integrated into the appropriate chromosome at the appropriate position for use in somatic cell nuclear transfer (see U.S. Pat. No. 5,945,577, herein incorporated by reference). Also desirable would be an embryonic stem cell line comprising the nucleic acid construct.

As discussed above, homogametic, egg-producing animals, i.e., female mammals, carrying the transgene on at least one sex chromosome, are carriers of the transgene and may be bred to propagate the trait. Accordingly, the present invention also encompasses methods for breeding a line of transgenic female mammals carrying a transgene on at least one sex chromosome. Such methods involve, essentially, testing female progeny of said line of transgenic female mammals for said transgene and using said transgene-positive female progeny to carry the line.

In such breeding methods, progeny may be generated using natural breeding techniques, thereby having one copy of said transgene. Alternatively, progeny may be generated from intracytoplasmic sperm transfer from a carrier male which produces substantially male progeny, thereby having two copies of said transgene.

The invention also encompasses transgenic female mammals produced by such breeding methods, and methods of using such transgenic female mammals for producing male mammals which produce substantially male progeny. Such a method comprises breeding the transgenic female mammals such that transgenic male mammals are produced. The transgenic male mammals thereby produced are also part of the invention.

As also described above, the transgene propagated in such breeding methods is expressed post-meiotically in spermatids produced by transgenic male progeny of said transgenic females. Where the transgene has a disabling effect on the spermatid, such transgenic male progeny produce substantially male offspring using natural breeding techniques. Alternatively, where the transgene confers a competitive advantage on the sperm, the resulting transgenic male progeny produce substantially female offspring using natural breeding techniques. The probability of having offspring of a single sex will vary depending on the nature of the transgene. However, progeny that are “substantially” either male or female is taken to mean almost always, to allow for the slim possibility that a disadvantaged transgenic sperm will fertilize an egg before a non-transgenic sperm, or for the slim possibility that transgenic sperm having a competitive advantage will lose to non-transgenic sperm.

Although it is believed that the present invention may be readily performed using any type of animal, preferred animals include mammals, which more preferably include mice, cows and pigs.

The full breadth of the invention will be further evident by reference to the following experimental methods.

### DETAILED DESCRIPTION OF THE PREFERRED EMBODIMENTS

### Transgene Constructs for Evaluating the Specificity of the Protamine Promoter

Two constructs were designed. The first construct was designed to test the function of the protamine promoter by pairing the promoter with an EGFP gene construct. Six transgenic mice were made (3 males and three females), and one of the males expressed EGFP that was localized to whole sperm (data not shown). The second construct was similar except that it contained a nuclear localization sequence. The objective is to determine whether EGFP expression could be localized to the nucleus of the sperm. Three transgenic mice were made and male offspring are to be produced so that sperm may be evaluated.

### Transgene Constructs for Evaluating the Transfer of Proteins Between Spermatids

The 85 and 27 kDa proteins of the outer dense fibers (ODF) of sperm, or derivatives thereof, may be expressed in developing sperm from the transgene in order to accomplish the methods of the invention. Sequence information is available for both of these proteins so it should be straightforward to (i) isolate a population of mouse spermatids, (ii) prepare a cDNA library for screening and (iii) screen such a library to obtain a cDNA clone or clones that can be used to make the transgenics or to PCR amplify the appropriate sequence. Constructs containing a fusion gene, incorporating green fluorescent protein (GFP) into the ODF protein sequence, may also be readily constructed using techniques known in the art. A description of these constructs is given below.

1. CMV/ODF−GFP+SV40 OR IRES/NEO: This construct will allow testing of the functionality of the ODF/GFP fusion protein cassette in fibroblasts.

2. CMV/NEO+PROT/ODF−GFP: This construct will allow selection in ES or fibroblast cells and later expression in spermatids with spermatid tracking of the GFP fluorescence. A functional PROT/ODF−GFP cassette may be used to prepare the construct for homologous recombination.

Fusion protein constructs will be valuable for identifying sperm carrying the transgene and for investigating the transfer of protein between spermatids. The promoter used in this construct is the mouse protamine promoter, but any promoter or other expression control element whose effect is to restrict gene expression to post-meiotic spermatids may be used.

Transgenic mice will be made with the constructs. At sexual maturity, the males will be paired with females and the transmission of the transgene will be monitored. In addition, transgenic females will be mated to produce transgenic males. These GI and GO males will be paired with females and the transmission of the transgene will be monitored. After mating at least five females, the males will be euthanized and the testis and the epididymus removed. A sperm sample will be taken from the epididymus and examined for the presence of GFP in half of the sperm. If these results are equivocal, then the testis will be cryosectioned to examine the seminiferous tubules and the distribution of GFP.

It is expected that the transgene will be passed to offspring from female transgenics but not from male transgenics. This would indicate that the transgenic protein affects fertility of the sperm. If not, it will be necessary to prepare a construct with the modified gene and retest the affect on fertility. (Note: no sex ratio alteration in offspring is expected because the transgene will not be targeted to the sex chromosomes in this study).

It is expected that the transgenic half of the sperm from transgenic males should fluoresce green in the tail due to expression of the green fluorescence fusion protein. This would indicate successful postmeiotic expression of the transgene. Furthermore, it would indicate correct localization of the transgene to only the transgenic half of the spermatids.

### Identification of a DNA Sequence on the X-Chromosome in Cattle that Could Be Used for Gene Targeting

As described previously, a deleterious gene inserted into the X-chromosome so that it will be expressed in X-bearing spermatids will reduce the fertility of the sperm that would give rise to female calves. This gene should be inserted into a site where it is likely to be expressed. To do this, a sequence adjacent to an endogenous gene that is expressed constitutively will be identified. Characterization of this DNA region is necessary to avoid disrupting gene function.

This may be accomplished by screening a bovine YAC library to isolate two Yeast Artificial Chromosome (YAC) clones containing tag sites that have been identified to be located in the X-chromosome specific region, near the pseudo-autosomal boundary (PAB) region. Fluorescent in situ hybridization (FISH) of the YAC clones will allow confirmation of localization to the appropriate site of the X-chromosome. The clones will be sub-cloned into a cosmid vector to derived smaller DNA inserts. Exon trapping will be used to identify the presence of coding sequences along the length of these cosmid clones.

To perform exon trapping, cosmid clones will be subcloned into the plasmid pSPL3. Subcloning is followed by transfection of subcloned DNA into COS-7 cells. After transient expression, RNA is harvested and reverse transcribed using a vector-specific oligonucleotide to yield first-strand cDNA. After digestion of the RNA template, an initial round of PCR is performed, followed by digestion with BstXI to remove PCR products that do not contain exons. A second round of PCR is performed, followed by rapid cloning into a phagemid vector using uracil DNA glycosylate.

Trapped exons will then be used to identify cosmid regions containing coding sequences. Some of these sequences will be used to screen bovine cDNA libraries and identified the full length genes to define the cosmid regions to be avoided for homologous recombination. Cosmid regions devoid of exons and repetitive sequences will be characterized for used as target sites for homologous recombination.

It is expected that the above techniques will allow the insertion of a vector construct by homologous recombination into an appropriate region of the X chromosome, such that insertion of the vector construct will not be deleterious to the transgenic animal. Similar methods could be performed with the Y chromosome, or autosomes.

### Insert the DNA Construct into the Previously Identified X-Chromosome Site and Select a Correctly Targeted Fibroblast Cell Line that can be Used for Nuclear Transfer

To correctly target a sequence in a primary cell line with a limited lifespan, large scale transfection, cloning and selection need to be done. Procedures for optimizing transfection efficiency and selecting and passaging transgenic clonal lines of cells are known in the art, and may be employed for this purpose.

Essentially, a DNA construct will be engineered by flanking the positive (CMV/neo) selectable marker and the gene of interest with the protamine promoter cassette with X-chromosome homologous sequences. The negative (SV40/Hyg) selectable marker will be located downstream of the 3′ homologous X-chromosome homologous sequence and will be deleted when homologous recombination occurs. The constructs will be as follows:

1. BTX5′ SEQUENCE+CMV/NEO+PROT/ODF−GFP+BTX3′ SEQUENCE+SV40/HGR.

2. BTX5′ SEQUENCE+PROT/ODF−GFP+CMV/NEO+BTX3′ SEQUENCE+SV40/HGR.

3. BTX5′ SEQUENCE+CMV/NEO+PROT/ODF+BTX3′ SEQUENCE+SV40/HGR.

4. BTX5′ SEQUENCE+PROT/ODF+CMV/NEO+BTX 3′ SEQUENCE+SV40/HGR.

The CMV/neo cassette allows selection for DNA insertion in fibroblasts. The PROT/ODF−GFP cassette will be expressed in spermatids and GFP allows visualization of expression. The PROT/ODF might be necessary if the fusion protein molecule is too big to move to its destination site and be assembled into the ODF. If this is the case the ODF proteins will need to be mutagenized as well. Since dicystronic constructs show reduced efficiency of expression of the 3′ cystron, both a construct with the CMV/neo+PROT/ODF order and another reversing this configuration should be initially tested.

Electroporation parameters may also be optimized using techniques well known in the art. Cells will then be grown in selectable media and surviving colonies will be propagated. A mix of the total population will then be evaluated by PCR to determine if homologous recombinants have been produced. If homologous recombinants are present then an initial serial dilution with each well containing about 10 cells in 500 wells will be grown up and evaluated by PCR. This will ensure that negative selection is only done on populations of cells that have homologous recombinants present. Therefore, any negatively selected clone that survives can be discarded. Any clone that dies following replica plating will be considered a true homologous recombinant and will be screened by Southern analysis. The cells that will be used will be fetal fibroblasts with a life span of about 35 population doublings. Population doublings will be monitored through the selection process to minimize and access the expected time of senescence. Approximately 3 to 5 cell lines will be frozen and shipped to Ultimate Biosystems for production of offspring.

It is expected that the first round of selection will go well in producing transgenic cells, and that the experiment can be easily replicated so that screening of many thousands of clones can be done readily. A preliminary screening will be done by PCR to identify homologous recombinants. Several recombinants should then be tested to identify those that grow well in culture.

What is claimed is:

1. A method for producing a mammal which has an altered tendency to produce progeny of a particular sex, comprising introducing a nucleic acid construct into at least one sex chromosome of the germ line of said mammal, wherein said nucleic acid construct encodes a transgene which is expressed post-meiotically in developing spermatids, and wherein expression of said transgene alters the fertility of sperm resulting from said developing spermatids, such that said mammal has an altered tendency to produce progeny of a particular sex.

2. The method of claim 1, wherein said mammal is heterogametic.

3. The method of claim 1, wherein said mammal is homogametic.

4. The method of claim 2, wherein said mammal has an altered tendency to produce first generation progeny having a particular sex.

5. The method of claim 3, wherein said mammal has an altered tendency to produce second generation progeny having a particular sex.

6. The method of claim 1, wherein said mammal is produced using nuclear transfer technology.

7. The method of claim 1, wherein said mammal is produced using natural breeding.

8. The method of claim 1, wherein said mammal is produced using intracytoplasmic sperm injection.

9. The method of claim 1, wherein said mammal is selected from the group consisting of mice, cows and pigs.

10. The method of claim 1, wherein expression of said transgene is controlled by a sperm-specific control sequence.

11. The method of claim 10, wherein said sperm-specific control sequence is a promoter selected from the group consisting of the protamine 1 or 2 gene promoters.

12. The method of claim 1, wherein said transgene is selected from the group consisting of sperm structural proteins, mutated variants thereof and fusion proteins designed therefrom.

13. The method of claim 12, wherein said sperm structural protein is an outer dense fiber (ODF) protein).

14. The method of claim 12, wherein said fusion protein comprises a fusion to green fluorescent protein (GFP).

15. The method of claim 1, wherein said mammal has an increased tendency to produce male progeny.

16. The method of claim 1, wherein said mammal has an increased tendency to produce female progeny.

17. The method of claim 5, wherein said second generation progeny are substantially male.

18. A transgenic mammal produced by the method of claim 1.

19. A line of transgenic mammals produced by breeding the mammal of claim 3.

20. A method for substantially altering the natural probability of producing progeny of a particular sex comprising breeding the transgenic mammal of claim 1 using natural breeding techniques such that the natural probability of producing progeny of a particular sex is substantially altered in any successive generation.

21. A nucleic acid construct comprising a sperm-specific control sequence operably linked to a cDNA sequence encoding a protein selected from the group consisting of sperm structural proteins, mutated versions thereof, and fusion proteins designed therefrom.

22. A vector comprising the nucleic acid construct of claim 21.

23. A fibroblast cell line comprising the nucleic acid construct of claim 21.

24. An embryonic stem cell comprising the nucleic acid construct of claim 21.

25. A method for breeding a line of transgenic female mammals carrying a transgene on at least one sex chromosome, wherein said transgene is expressed post-meiotically in spermatids produced by transgenic male progeny of said transgenic females, such that said transgenic male progeny produce substantially male offspring using natural breeding techniques, said method comprising testing female progeny of said line of transgenic female mammals for said transgene and using said female progeny to carry the line.

26. The method of claim 25, wherein said female progeny are generated using natural breeding techniques, and have one copy of said transgene.

27. The method of claim 25, wherein said female progeny are generated from intracytoplasmic sperm transfer from a carrier male which produces substantially male progeny, said female progeny having two copies of said transgene.

28. A transgenic female mammal produced by the method of claim 25.

29. A method of producing a male mammal which produces substantially male progeny comprising breeding the transgenic female mammal of claim 28 such that a transgenic male mammal is produced.

30. A transgenic male mammal produced by the method of claim 29.
